(19) United States
(12) Patent Application Publication
(10) Pub. No.: US 2025/0134566 A1
(43) Pub. Date: May 1, 2025

(54) POLYETHERETHERKETONE CARBON FIBER COMPOSITE BONE FUSION PLATE

(71) Applicant: In2Bones USA, LLC, Memphis, TN (US)

(72) Inventors: Bradley Cowan, Memphis, TN (US); Rebecca Hawkins Wahl, Escondido, CA (US); Alan Taylor, Memphis, TN (US)

(73) Assignee: In2Bones USA, LLC, Memphis, TN (US)

(21) Appl. No.: 18/384,686

(22) Filed: Oct. 27, 2023

(51) Int. Cl. A61B 17/80; A61B 17/17; A61B 17/56

(52) U.S. Cl. CPC A61B 17/8057; A61B 17/1728; A61B 2017/564

(57) ABSTRACT

An apparatus and methods are provided for a PEEK carbon fiber bone fusion plate for stabilizing and fixating fractures, revision procedures, joint fusion, osteotomies and reconstruction of the small bones of the human body. The PEEK carbon fiber bone fusion plate comprises a generally elongated member having an upper surface and a lower bone contact surface. The bone fusion plate includes a composite structure comprising PEEK-embedded carbon fiber tape oriented at different angles. Two or more fixation apertures are disposed along the bone fusion plate and configured to receive bone screws. Each of the fixation apertures includes a threaded hole configured to engage with a head of a locking or non-locking bone screw.

### FIELD

Embodiments of the present disclosure generally relate to bone securing devices. More specifically, embodiments of the disclosure relate to an apparatus and methods for stabilization and fixation of fractures, revision procedures, joint fusion, osteotomies and reconstruction of the small bones of the human body such as in the hand, wrist, foot, and ankle.

### BACKGROUND

A fusion bone plate implant may be utilized in conjunction with one or more fasteners so as to generate compression and stability at a bone interface. An implant coupled with fasteners generally serves to stabilize bones, or bone parts, relative to one another so as to promote bone fusion. In many applications, bone plates and fasteners are used to fuse bones, or bone parts, of the human body, such as bones in the foot, the ankle, the hand, the wrist, as well as various other portions of the body. Furthermore, during the course of certain medical procedures, a surgeon may immobilize one or more bones or the bone fragments by stabilizing the bones together in a configuration which approximates the natural anatomy. To this end, the surgeon may use fasteners to attach the bones to a bone plate implant so as to hold the bones in alignment with one another while they fuse together.

### SUMMARY

An apparatus and methods are provided for a Polyetheretherketone (PEEK) carbon fiber composite bone fusion plate for stabilizing and fixating fractures, revision procedures, joint fusion, osteotomies and reconstruction of the small bones of the human body such as in the hand, wrist, foot, and ankle. The bone fusion plate comprises a generally elongated member having an upper surface and a lower bone contact surface. The bone fusion plate is composed of a PEEK carbon fiber reinforced composite material which includes a series of layers of PEEK-embedded carbon fibers oriented at different angles. Two or more fixation apertures are disposed along the bone fusion plate and configured to receive bone screws. Each of the fixation apertures includes a threaded hole configured to engage with either a threaded (locking) head or non-threaded head (non-locking) bone screw. When a threaded head screw is assembled it provides a rigid plate/screw construct. When a non-locking screw is assembled to the plate, it can be placed at a variable angle to the plate surface. The threaded hole can be tapered or non-tapered.

In an exemplary embodiment, an apparatus for stabilizing and fixating fractures, revision procedures, joint fusion, osteotomies and reconstruction of the small bones of the human body comprises: a bone fusion plate comprising a generally elongate member having an upper surface and a lower, bone contact surface; a composite material comprising a series of layers of PEEK-embedded carbon fibers oriented at different angles; two or more fixation apertures disposed along the bone fusion plate and configured to receive bone screws; a threaded hole comprising each of the two or more fixation apertures; and at least one non-threaded elongated aperture configured to receive a bone screw.

In another exemplary embodiment, the two or more fixation apertures comprise 4-5 fixation apertures. In another exemplary embodiment, the two or more fixation apertures comprise more than 5 fixation apertures.

In another exemplary embodiment, the threaded hole is tapered and the included taper angle ranges between about 10 degrees and about 120 degrees. In another exemplary embodiment, the taper angle is either about 25 degrees or about 45 degrees. In another exemplary embodiment, the threaded hole surface is configured to engage with the head of a locking bone screw. In another exemplary embodiment, the threaded hole surface is configured to engage with the head of a non-locking bone screw.

In another exemplary embodiment, the threaded hole is configured to accept a threaded locking drill guide tower for on-axis pilot hole preparation. In another exemplary embodiment, the threaded hole is configured to substantially reduce occurrences of debris due to screw assembly. In another exemplary embodiment, the threaded hole is configured to advantageously eliminate instances of cold welding.

In another exemplary embodiment, the elongated aperture is configured to receive the bone screw at an angle of substantially 90° with respect to the plane of the bone fusion plate. In another exemplary embodiment, the elongated aperture is configured to receive the bone screw at an oblique angle with respect to the plane of the bone fusion plate.

In another exemplary embodiment, the layers of PEEK carbon fiber tape are compression molded together into the shape of the plate. In another exemplary embodiment, different angles of the carbon fiber layers are configured to optimize the strength of bone fusion plate and how it is stressed in the body. In another exemplary embodiment, the PEEK carbon fiber layers comprising the bone fusion plate are configured to provide slight plate visualization on X-RAY without obscuring the fusion site.

In another exemplary embodiment, the bone fusion plate comprises configuring a plate length that ranges between about 0.90 inches and about 10.00 inches. In another exemplary embodiment, the bone fusion plate comprises configuring a plate width that ranges between about 0.25 inches and about 2.00 inches. In another exemplary embodiment, the bone fusion plate comprises configuring a plate thickness that ranges between about 0.04 inches and about 0.10 inches.

In another exemplary embodiment, a longitudinal aspect of the bone fusion plate includes one or more angles or bends such that the bone fusion plate matches the anatomy of the bone to be fused. In another exemplary embodiment, the bone fusion plate includes a vertical angle configured to accommodate a dorsiflexion angle of the bones in a human foot. In another exemplary embodiment, the vertical angle ranges between about 2 degrees and about 10 degrees.

In another exemplary embodiment, the bone fusion plate includes a horizontal angle configured to accommodate a hallux valgus angle of the human foot. In another exemplary embodiment, the horizontal angle ranges between about 2 degrees and about 10 degrees. In another exemplary embodiment, a transverse aspect of the bone fusion plate includes one or more curvatures such that the bone fusion plate matches the anatomy of the bone to be fused.

In another exemplary embodiment, the bone fusion plate includes any of various markings, shapes, diagrams, symbols, words, numbers, or indicators configured to assist a practitioner with correctly orienting the bone fusion plate onto the bones to be treated. In another exemplary embodiment, the bone fusion plate includes a joint line indicator disposed on the upper surface to assist the practitioner with aligning a longitudinal aspect of the bone fusion plate with a joint to be fixated.

In another exemplary embodiment, the bone fusion plate is configured to be shipped and stored in a sterile state within a sterile packaging. This packaging can be either single barrier or dual barrier. The packaging material is capable of without-sterilization methods such as GAMMA, Ethylene Oxide, Steam, and Electron Beam. In another exemplary embodiment, the sterile packaging consists of a tube package comprising an inner tube contained inside an outer tube. In another exemplary embodiment, the outer tube is configured to receive and retain the inner tube, such that a sterile environment within at least the inner tube is maintained during shipping and storage of the fusion bone plate. In another exemplary embodiment, the inner tube and the outer tube comprise a rigid material that is capable of withstanding sterilization by way of GAMMA Irradiation.

In another exemplary embodiment, the inner tube is configured to house an internal implant retainer that is configured to protect the bone fusion plate within the inner tube during shipping and storage inside the sterile tube package. In another exemplary embodiment, the internal implant retainer comprises: a first half and a second half that share intervening fold lines; and a retaining cavity and one or more push clasps disposed in each of the first half and the second half. In another exemplary embodiment, the first half is configured to be folded onto to the second half, by way of the fold lines, to surround the bone fusion plate between the retaining cavities. In another exemplary embodiment, the push clasps are configured to be pressed together, such that the bone fusion plate is enclosed within the retaining cavities for storage and shipping within the sterile package.

In an exemplary embodiment, an apparatus for stabilizing and fixating fractures, revision procedures, joint fusion, osteotomies and reconstruction of the bones of the human body comprises: a bone fusion plate comprising a generally elongate member having an upper surface and a lower, bone contact surface; a layer structure comprising a series of layers of PEEK-embedded carbon fiber tape oriented at different angles; two or more fixation apertures disposed along the bone fusion plate and configured to receive bone screws; and a threaded hole comprising each of the two or more fixation apertures; wherein a plate length ranges between about 0.90 inches and about 10.00 inches, a plate width ranges between about 0.25 inches and about 2.00 inches, and a plate thickness ranges between about 0.04 inches and about 0.10 inches.

In another exemplary embodiment, the bone fusion plate includes at least one elongated aperture configured to receive a bone screw. In another exemplary embodiment, the threaded hole is tapered. In another exemplary embodiment, the threaded hole is non-tapered. In another exemplary embodiment, the threaded hole is configured to engage with the head of a locking bone screw. In another exemplary embodiment, the threaded hole is configured to engage with the head of a non-locking bone screw. In another exemplary embodiment, the threaded hole is configured to accept a threaded locking drill guide tower for on-axis pilot hole preparation.

In another exemplary embodiment, the PEEK-embedded carbon fiber tape includes an additional additive that provides visualization of both the fusion site and the bone plate under X-Ray. In another exemplary embodiment, the additive material is Barium Sulfate. In another exemplary embodiment, a longitudinal aspect of the bone fusion plate includes one or more angles or bends such that the bone fusion plate matches the anatomy of the bone to be fused. In another exemplary embodiment, the bone fusion plate includes a vertical angle configured to accommodate a dorsiflexion angle of the bones in a human foot. In another exemplary embodiment, the bone fusion plate includes a horizontal angle configured to accommodate a hallux valgus angle of the human foot ranging between about 2 degrees and about 10 degrees.

In another exemplary embodiment, a transverse aspect of the bone fusion plate includes one or more curvatures such that the bone fusion plate matches the anatomy of the bone to be fused. In another exemplary embodiment, the bone fusion plate includes any of various markings, shapes, diagrams, symbols, words, numbers, or indicators configured to assist a practitioner with correctly orienting the bone fusion plate onto the bones to be treated. In another exemplary embodiment, the bone fusion plate includes a joint line indicator disposed on the upper surface to assist the practitioner with aligning a longitudinal aspect of the bone fusion plate with a joint to be fixated.

In another exemplary embodiment, the bone fusion plate is configured to be shipped and stored in sterile packaging in a single barrier or dual barrier configuration. In another exemplary embodiment, the sterile packaging is a tube assembly comprising an inner tube contained inside an outer tube. In another exemplary embodiment, the inner tube is configured to house an internal implant retainer that is configured to protect the bone fusion plate within the sterile packaging during shipping and storage. In another exemplary embodiment, the internal implant retainer comprises: a first half and a second half that share intervening fold lines; and a retaining cavity and one or more push clasps disposed in each of the first half and the second half. In another exemplary embodiment, the first half is configured to be folded onto to the second half, by way of the fold lines, to surround the bone fusion plate between the retaining cavities; and wherein the push clasps are configured to be pressed together, such that the bone fusion plate is enclosed within the retaining cavities for storage and shipping within the sterile package.

In another exemplary embodiment, the sterile packaging is a thermoform tray assembly comprising an inner tray contained inside an outer tray. In another exemplary embodiment, the sterile packaging is a pouch assembly comprising an inner pouch contained inside an outer pouch. In another exemplary embodiment, the sterile packaging is capable of withstanding sterilization by way of GAMMA sterilization. In another exemplary embodiment, the sterile packaging is capable of withstanding sterilization by way of Ethylene Oxide Sterilization. In another exemplary embodiment, the sterile packaging is capable of withstanding sterilization by way of Steam sterilization. In another exemplary embodiment, the sterile packaging is capable of withstanding sterilization by way of Electron Beam sterilization.

In an exemplary embodiment, a method for stabilizing and fixating fractures, revision procedures, joint fusion, osteotomies and reconstruction of the bones of the human body comprises: configuring a bone fusion plate comprising a generally elongate member having an upper surface and a lower, bone contact surface; forming a layer structure comprising the bone fusion plate; disposing two or more fixation apertures along the bone fusion plate; configuring the two or more fixation apertures to receive bone screws; configuring a threaded hole comprising each of the two or more fixation apertures; and configuring at none or least one elongated aperture to receive a bone screw.

In another exemplary embodiment, disposing the two or more fixation apertures comprises disposing 4-5 fixation apertures. In another exemplary embodiment, disposing the two or more fixation apertures comprises disposing more than 5 fixation apertures.

In another exemplary embodiment, configuring the threaded hole includes disposing the threaded hole below the upper surface of the bone fusion plate. In another exemplary embodiment, configuring the threaded hole includes configuring the threaded hole to allow a countersunk head of the bone screw to assume a level that is flush with, or disposed below, the upper surface when the fastener is tightened to hold the bone fusion plate against the bone.

In another exemplary embodiment, configuring the threaded and tapered hole includes forming an included taper angle ranging between about 10 degrees and 120 about degrees with respect to the upper surface. In another exemplary embodiment, forming the taper angle includes forming the included angle comprising either about 25 degrees or about 45 degrees.

In another exemplary embodiment, configuring the threaded hole includes configuring the threaded hole to accept a threaded locking drill guide tower for on-axis pilot hole preparation. In another exemplary embodiment, configuring the elongated aperture includes configuring the elongated aperture to receive the bone screw at an angle of substantially 90° with respect to the plane of the bone fusion plate. In another exemplary embodiment, configuring the elongated aperture includes configuring the elongated aperture to receive the bone screw at an oblique angle with respect to the plane of the bone fusion plate.

In another exemplary embodiment, the carbon fiber tape layers are oriented at varying angles to optimize strength in anticipated loading directions. In another exemplary embodiment, configuring the bone fusion plate includes configuring a longitudinal aspect of the bone fusion plate to include one or more angles or bends such that the bone fusion plate matches the anatomy of the bone to be fused. In another exemplary embodiment, configuring the bone fusion plate comprises configuring a vertical angle to accommodate a dorsiflexion angle of the bones in a human foot, the vertical angle ranging between about 2 degrees and about 10 degrees. In another exemplary embodiment, configuring the bone fusion plate includes configuring a horizontal angle to accommodate a hallux valgus angle of the human foot, the horizontal angle ranging between about 2 degrees and about 10 degrees. In another exemplary embodiment, configuring the bone fusion plate includes configuring a transverse aspect of the bone fusion plate to include one or more curvatures such that the bone fusion plate matches the anatomy of the bone to be fused.

In another exemplary embodiment, configuring the bone fusion plate includes configuring any of various markings, shapes, diagrams, symbols, words, numbers, or indicators to assist a practitioner with correctly orienting the bone fusion plate onto the bones to be treated. In another exemplary embodiment, configuring the bone fusion plate includes disposing a joint line on the upper surface to assist the practitioner with aligning a longitudinal aspect of the bone fusion plate with a joint to be fixated.

In another exemplary embodiment, configuring the bone fusion plate includes configuring the bone fusion plate to be shipped and stored in a sterile state. In another exemplary embodiment, configuring the bone fusion plate to be shipped and stored in a sterile state includes forming a sterile tube package comprising an inner tube contained inside an outer tube. In another exemplary embodiment, forming the sterile tube package includes configuring the outer tube to receive and retain the inner tube, such that a sterile environment within at least the inner tube is maintained during shipping and storage of the fusion bone plate. In another exemplary embodiment, forming the sterile tube package includes forming the inner tube and the outer tube of a rigid material that is capable of withstanding sterilization by way of GAMMA Irradiation. In another exemplary embodiment, forming the sterile tube package includes configuring the inner tube to house an internal implant retainer.

In another exemplary embodiment, the sterile tube package includes configuring the internal implant retainer to protect the bone fusion plate within the inner tube during shipping and storage inside the sterile tube package. In another exemplary embodiment, configuring the internal implant retainer comprises: forming a first half and a second half that share intervening fold lines; and disposing a retaining cavity and one or more push clasps in each of the first half and the second half. In another exemplary embodiment, forming the first half includes configuring the first half to be folded onto to the second half, by way of the fold lines, to surround the bone fusion plate between the retaining cavities. In another exemplary embodiment, disposing the push clasps includes configuring the push clasps to be pressed together, such that the bone fusion plate is enclosed within the retaining cavities for storage and shipping within the sterile package.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an upper perspective view of an exemplary embodiment of a PEEK carbon fiber bone fusion plate, according to the present disclosure;

FIG. 2 illustrates an exploded view of an exemplary embodiment of a layered structure that may be incorporated into a PEEK carbon fiber bone fusion plate in accordance with the present disclosure;

FIG. 3 illustrates a close-up view of an exemplary embodiment of a threaded aperture comprising a PEEK carbon fiber bone fusion plate, according to the present disclosure;

FIG. 4 illustrates a dimensioned view of an exemplary embodiment of a PEEK carbon bone fusion plate in accordance with the present disclosure;

FIG. 5 illustrates an exemplary embodiment of a PEEK carbon bone fusion plate that includes an angulation range that is configured to accommodate a proper dorsiflexion angle;

FIG. 6 illustrates an exemplary embodiment of a PEEK carbon fiber bone fusion plate that includes an angulation range that is configured to accommodate a proper hallux valgus angle;

FIG. 7 illustrates an exemplary embodiment of a PEEK carbon fiber bone fusion plate that includes a curvature that is configured to accommodate the anatomy of an indicated area;

FIG. 8 illustrates an exemplary embodiment of a PEEK carbon fiber bone fusion plate that includes a joint line indicator configured to assist a practitioner with aligning the plate with a joint to be fixated;

FIG. 9 illustrates an exemplary embodiment of a sterile tube package for shipping and storing the PEEK carbon fiber bone fusion plate in a sterile state, according to the present disclosure;

FIG. 10 illustrates an exemplary embodiment of an internal implant retainer configured to protect the PEEK carbon fiber bone fusion plate during shipping and storage inside a sterile tube package in accordance with the present disclosure;

FIG. 11 illustrates an exemplary-use environment wherein a practitioner is withdrawing an inner tube from an outer tube comprising an exemplary embodiment of a sterile tube package, according to the present disclosure;

FIG. 12 illustrates an exemplary-use environment wherein the practitioner is withdrawing an internal implant retainer from within an inner tube comprising an exemplary embodiment of a sterile tube package, in accordance with the present disclosure;

FIG. 13 illustrates an exemplary-use environment wherein the practitioner is opening an exemplary embodiment of an internal implant retainer and accessing an exemplary embodiment of a PEEK carbon fiber bone fusion plate from within the internal implant retainer in accordance with the present disclosure;

FIG. 14 illustrates a top view of an exemplary embodiment of a PEEK carbon fiber bone fusion plate, according to the present disclosure;

FIG. 15 illustrates a longitudinal view of the bone fusion plate of FIG. 14, showing a transverse curvature that is configured to accommodate the anatomy of an indicated area;

FIG. 16 illustrates a top view of an exemplary embodiment of a PEEK carbon fiber bone fusion plate, according to the present disclosure;

FIG. 17 illustrates a cross-sectional view of the bone fusion plate of FIG. 16, taken along a midline, in accordance with the present disclosure;

FIG. 18 illustrates a top view of an exemplary embodiment of a PEEK carbon fiber bone fusion plate that includes an angulation range configured to accommodate a proper hallux valgus angle of a patient's left foot, according to the present disclosure;

FIG. 19 illustrates a top view of an exemplary embodiment of a PEEK carbon fiber bone fusion plate that includes an angulation range configured to accommodate a proper hallux valgus angle of a patient's right foot, in accordance with the present disclosure;

FIG. 20 illustrates a midline cross-sectional view of the bone fusion plate of FIG. 18, showing an angulation range that is configured to accommodate a proper dorsiflexion angle; and

FIG. 21 illustrates a cross-sectional view of the bone fusion plate of FIG. 19, taken along a midline, in accordance with the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the PEEK carbon fiber bone fusion plate and methods disclosed herein may be practiced without these specific details. In other instances, specific numeric references such as “first plate,” may be made. However, the specific numeric reference should not be interpreted as a literal sequential order but rather interpreted that the “first plate” is different than a “second plate.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In general, the present disclosure describes an apparatus and methods for stabilization and fixation of fractures, revision procedures, joint fusion, osteotomies and reconstruction of the small bones of the human body such as bones in the hand, wrist, foot and ankle (e.g., the talus and tibial bones). The apparatus comprises a bone fusion plate which comprises an elongate member having one or more fixation apertures and one or more elongated apertures. The bone fusion plate comprises Polyetheretherketone (PEEK) carbon fiber, possessing a tensile strength suitable for immobilizing bones. The fixation apertures are configured to receive fasteners suitable for fastening the bone fusion plate to the bones to be fixated. Each of the fixation apertures includes a threaded hole disposed below an upper surface of the bone fusion plate. The threaded hole is configured to engage with locking as well as non-locking fasteners. The elongated aperture is not threaded may receive a fastener at an oblique angle for compressing adjacent bone portions together so as to encourage bone fusion. The bone fusion plate includes one or more directions of curvature and one or more angulations configured to match the anatomy of the bones to the fused.

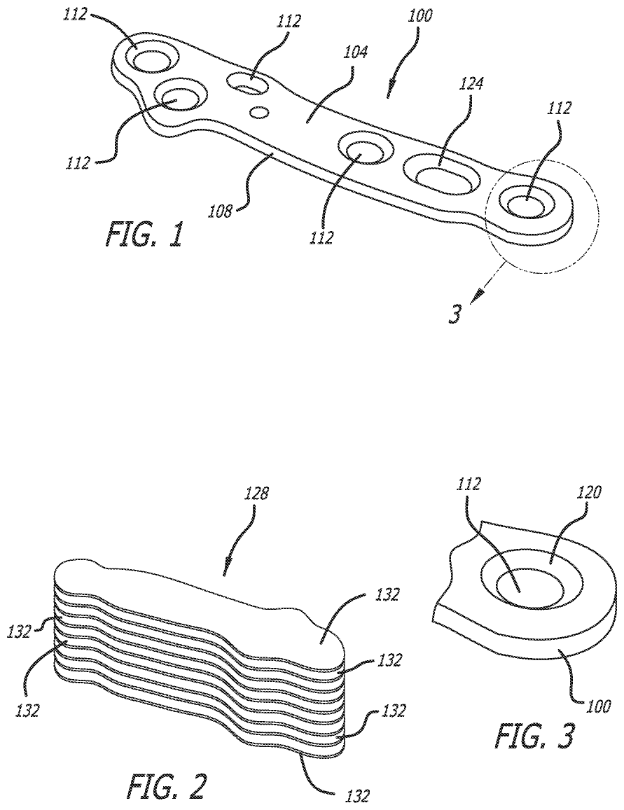

FIG. 1 illustrates an exemplary embodiment of a PEEK carbon fiber bone fusion plate 100 (hereinafter, “bone fusion plate 100”) configured for stabilization and fixation of fractures, revision procedures, joint fusion, osteotomies and reconstruction of the bones of the human body, according to the present disclosure. Bone fusion plate 100 comprises a generally elongated member having an upper surface 104 and a lower, bone contact surface 108. Disposed along the bone fusion plate 100 are two or more fixation apertures 112 suitable for receiving fasteners, such as bone screws 116 (see FIGS. 6-8). In some embodiments, the bone fusion plate 100 may include 4-5 fixation apertures 112, although in other embodiments the bone fusion plate may include less than 4 or more than 5 fixation apertures 112, without limitation.

As best shown in FIG. 3, the fixation aperture 112 includes a threaded hole 120 disposed below the upper surface 104 of the bone fusion plate 100. As will be appreciated, the threaded hole 120 allows a head of a bone screw 116 to assume a level that is flush with, or disposed below, the upper surface 104 when the fastener is tightened to hold the bone fusion plate 100 against the bone. In some embodiments, the threaded hole 120 may be non-tapered or tapered implemented with any of various suitable included angles, including, but not limited to, 60°, 82°, 90°, 100°, 110°, or 120° with respect to the upper surface 104. In some embodiments, however, the included taper angle of the threaded hole 120 preferably is either 82° or 90°. Further, it should be borne in mind that the threaded hole 120 is configured to engage with locking bone screws 116 and thus provides a locking feature that operates to prevent the bone screw 116 from backing out of the bone after having been implanted in a patient.

Moreover, in addition to accepting both locking and non-locking screws 116, the threaded holes 120 may be configured to accept a threaded locking drill guide tower for on-axis pilot hole preparation. Further, it is contemplated that the threaded holes (screw holes) 120 will substantially reduce occurrences of debris due to screw assembly. It is further contemplated that the threaded holes 120 will advantageously eliminate instances of cold welding.

As shown in FIG. 1, the bone fusion plate 100 preferably comprises at least one elongated aperture 124 configured to receive a threaded fastener, such as a bone screw 116 (see FIGS. 6-8). Similar to the fixation apertures 112, the elongated aperture 124 may receive the bone screw 116 at an angle of substantially 90° with respect to the plane of the bone fusion plate 100. Unlike the fixation apertures 112, however, the elongated aperture 124 may further receive the bone screw 116 at an oblique angle with respect to the plane of the bone fusion plate 100. The oblique angle of the bone screw 116 facilitates compressing adjacent bone portions together so as to encourage bone fusion. The bone screw 116 may be any component of hardware having a head configured to abut the surface of bone fusion plate 100 and a shaft configured to secure bone portions together in a fixed configuration. In some embodiments, the bone screw 116 may comprise a lag screw which includes a head that is rounded or tapered and is coupled to a shaft having an unthreaded portion adjacent to the head and a threaded portion that ends at a tip.

FIG. 2 illustrates an exploded view of an exemplary embodiment of a layer structure 128 that may be incorporated into the PEEK carbon fiber bone fusion plate 100, in accordance with the present disclosure. The layer structure 128 comprises a series of layers 132 comprising carbon fibers that can be oriented at different angles with varying layers of PEEK carbon fiber tape. As will be appreciated, the orientation of different angles of the carbon fibers comprising layers 132 optimizes mechanical properties, such as bending and fatigue strength, of the bone fusion plate 100. Further, the 100% homogenous PEEK carbon fiber composite comprising the bone fusion plate 100 provides X-ray transparency. Thus, the PEEK carbon fiber composite provides visualization of the bone fusion site without obstruction due to the presence of the plate 100. If visualization of the bone fusion plate 100 is also required, however, a radiopaque additive may be incorporated into the bone fusion plate 100. It is contemplated that a radiopaque additive such as Barium Sulfate in the right amount provides both visibility of the fusion site and the plate implant due to a “ghosting effect”.

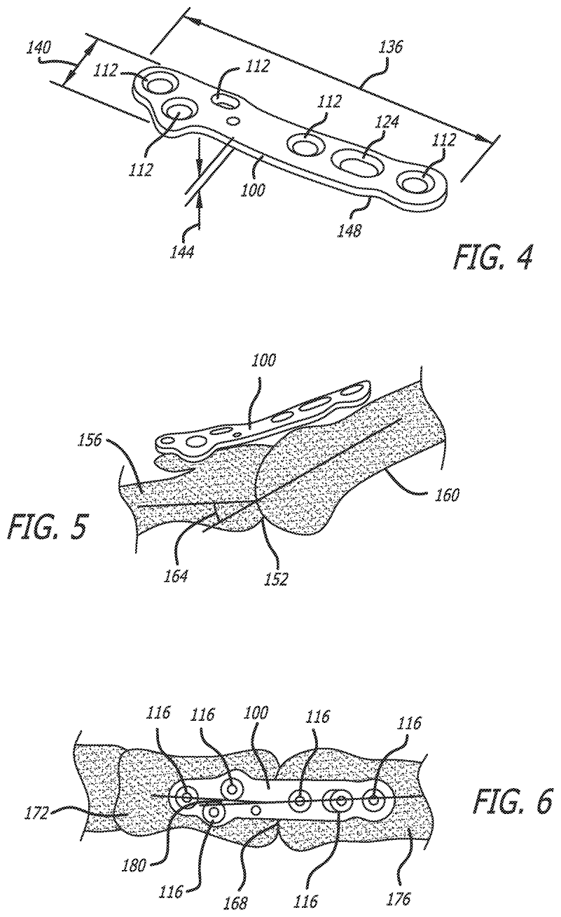

FIG. 4 illustrates a dimensioned view of the bone fusion plate 100 in accordance with the present disclosure. As mentioned herein, the overall dimensions of the bone fusion plate 100 may be varied according to the anatomy of the specific bones to be treated. To this end, in some embodiments, the bone fusion plate 100 comprises a plate length 136 that ranges between about 0.90 inches and about 10.00 inches. In some embodiments, the bone fusion plate 100 comprises a plate width 140 that ranges between about 0.25 inches and about 2.00 inches. In some embodiments, the bone fusion plate 100 comprises a plate thickness 144 that ranges between about 0.04 inches and about 0.10 inches. As further shown in FIG. 4, the bone fusion plate 100 can have straight sides or any of various surface features, such as one or more scallops 148 or other similar features that accommodate the anatomy of the specific bone being treated.

As shown in FIGS. 5-6, a longitudinal aspect of the bone fusion plate 100 can be characterized by one or more angles or bends such that the bone fusion plate 100 matches the anatomy of the bone to be fused. For example, as shown in FIG. 5, a bone fusion plate 100 configured to fixate a joint 152 between a first bone 156 and a second bone 160 can be curved in a vertical direction giving rise to a vertical angle 164. As will be appreciated, vertical angle 164 may be configured to accommodate a dorsiflexion angle of the bones in a human foot, and thus the vertical angle 164 may range between about 2 degrees and about 10 degrees. In another example, shown in FIG. 6, a bone fusion plate 100 configured is fastened across a joint 168 between a first bone 172 and a second bone 176 by way of multiple bone screws 116. The bone fusion plate 100 shown in FIG. 6 is curved in a horizontal direction giving rise to a horizontal angle 180 relative to the longitudinal aspect of the plate. As will be appreciated, the horizontal angle 180 may be configured to accommodate a hallux valgus angle of the human foot, and thus the horizontal angle 180 may range between about 2 degrees and about 10 degrees, without limitation.

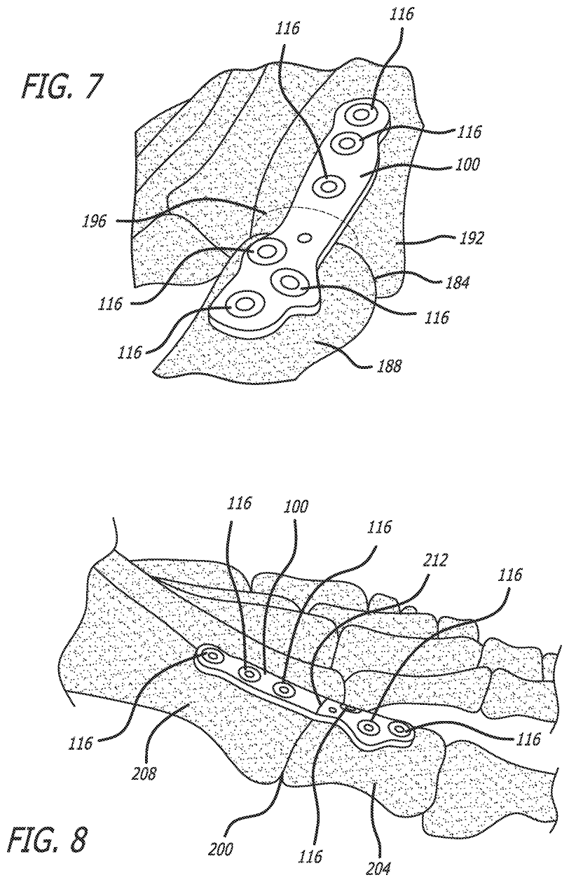

It is contemplated that the bone fusion plate 100 is not limited to bends or angles along the longitudinal aspect of the plate, but rather the bone fusion plate 100 may comprise curvature along a transverse aspect of the bone fusion plate 100. For example, as shown in FIG. 7, a bone fusion plate 100 is fastened across a joint 184 between a first bone 188 and a second bone 192 by way of multiple bone screws 116. The bone fusion plate 100 shown in FIG. 7 is curved in a direction that is substantially perpendicular to the longitudinal aspect of the plate, giving rise to a transverse curvature 196. As will be appreciated, the transverse curvature 196 may be configured to accommodate the specific anatomy of the bones 188, 192 to be fused.

It should be borne in mind that the bone fusion plate 100 is not limited to the specific angles and/or curvatures shown in the figures, but rather any combination of curvatures and/or angles may be disposed along the longitudinal and transverse aspects of the bone fusion plate 100, without limitation. In some embodiments, the curvatures incorporated into the longitudinal and transverse aspects of the bone fusion plate 100 may be configured to change as a function of distance from a middle of the bone fusion plate 100.

In some embodiments, the curvatures and/or angles along the longitudinal and transverse aspects of the bone fusion plate 100 may be configured to match a specific anatomy of the bones to be treated. As will be appreciated, a bone fusion plate 100 that is “anatomy specific” must be correctly fastened to the bones to be treated. To this end, the bone fusion plate 100 may include any of various markings, shapes, diagrams, symbols, words, numbers, or indicators configured to assist a practitioner with correctly orienting the bone fusion plate 100 onto the bones to be treated. In one exemplary embodiment, shown in FIG. 8, a bone fusion plate 100 is fastened across a joint 200 between a first bone 204 and a second bone 208 by way of multiple bone screws 116. As further shown in FIG. 8, the bone fusion plate 100 includes a joint line 212 disposed on an upper surface 104 of the plate 100. The joint line 212 assists the practitioner with aligning the longitudinal aspect of the bone fusion plate 100 with the joint 200, thereby ensuring that any curvatures and/or angles comprising the bone fusion plate 100 substantially match the specific anatomy of the first and second bones 204, 208. As will be appreciated, the joint line 212 simplifies accurately matching the curvatures and/or angles comprising the bone fusion plate 100 with the anatomy of the bones being treated.

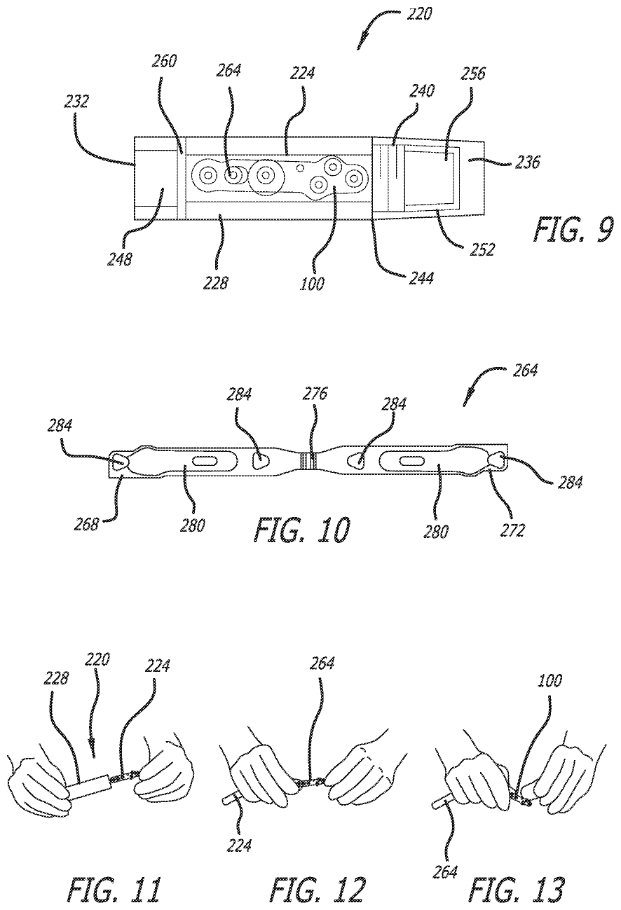

FIG. 9 illustrates an exemplary embodiment of a sterile tube package 220 for shipping and storing a PEEK carbon bone fusion plate 100 in a sterile state, according to the present disclosure. In the illustrated embodiment, the sterile tube package 220 comprises an inner tube 224 contained inside an outer tube 228. The outer tube 228 is configured to receive and retain the inner tube 224, such that a sterile environment within at least the inner tube 224 is maintained during shipping and storage of the bone fusion plate 100. The tubes 224, 228 may, in some embodiments, be circular or oval in cross-sectional shape, without limitation. Preferably, the tubes 224, 228 comprise a rigid material that is capable of withstanding sterilization by way of GAMMA Irradiation, such as, by way of example, any of clear plastic, various polymer compounds, and the like, without limitation.

The outer tube 228 generally is an elongated hollow member having an enclosed end 232 and an open end configured to be joined with a closure 236 by way of threads 240. The threads 240 comprise one or more circumferentially disposed threads on an exterior surface of the outer tube 228 surrounding the open end. The circumferentially disposed threads are configured to rotatably engage with similar threads disposed within the closure 236. In some embodiments, a seal 244 may be disposed around the open end and configured to establish an airtight coupling with the closure 236. As will be appreciated, inserting the inner tube 224 into the outer tube 228 and then rotatably engaging the closure 236 with the threads 240 until the seal 244 contacts the interior of the closure 236 secures the inner tube 224 within the outer tube 228. In some embodiments, a textured surface may be disposed on an exterior surface of the closure 236 so as to enable gripping and tightening the closure 136 by hand.

Moreover, in some embodiments, a support 248 may be disposed inside the enclosed end 232 of the outer tube 228 and configured to advantageously support a first end of the inner tube 224 once the closure 236 is fully engaged with the threads 240. Further, an interior 252 of the closure 236 may be configured to support a second end of the inner tube 224 once the closure 236 is fully engaged with the threads 240. It is contemplated that the support 248 and the interior 252 of the closure 236 cooperate to secure the inner tube 224 within the outer tube 228.

The inner tube 224 generally is an elongated hollow member having an enclosed end 256 and an open end configured to be joined with a closure 260. In some embodiments, the closure 260 may be configured to couple with the inner tube 224 by way of threads that are similar to the abovementioned threads 240. The threads may comprise one or more circumferentially disposed threads on an exterior surface of the inner tube 224 surrounding the open end. The circumferentially disposed threads may be configured to rotatably engage with similar threads disposed within the closure 260. Further, a seal similar to the seal 244 may be disposed around the open end of the inner tube 224 and configured to establish an airtight coupling with the closure 260. As such, tightening the closure 260 onto the inner tube 224 and then inserting the inner tube 224 into the outer tube 228, such that the closure 260 is in contact with the support 248, followed by tightening the closure 236 onto the outer tube 228, as described above, advantageously preserves the sterile environment within at least the inner tube 224.

It is contemplated that the inner tube 224 may be configured to house the bone fusion plate 100 in any of a variety of different configurations that are capable of preserving the sterile environment within the inner tube 224 and protecting the bone fusion plate 100 during shipping and storage. In one exemplary embodiment, illustrated in FIG. 10, an internal implant retainer 264 is configured to protect the bone fusion plate 100 within the inner tube 224 during shipping and storage inside the sterile tube package 220. The internal implant retainer 264 is generally of a clamshell variety of packaging that includes a first half 268 and a second half 272 that share intervening fold lines 276. A retaining cavity 280 and one or more push clasps 284 are disposed in each of the halves 268, 272. As will be appreciated, the first half 268 may be folded onto to the second half 272, by way of the fold lines 276, to surround the bone fusion plate 100 between the retaining cavities 280. Upon pressing the push clasps 284 together, the bone fusion plate 100 is enclosed within the retaining cavities 280 for storage and shipping within the sterile package 220.

Based on the foregoing description, it should be recognized that the sterile tube package 220 and the internal implant retainer 264 are advantageously configured to preserve the sterility of the fusion bone plate 100, or other surgical implements, during shipping and storage. During operation of the sterile tube package 220 and the internal implant retainer 264, a practitioner may turn the closure 236 to loosen the threads 240 and then remove the closure 236 from the open end of the outer tube 228. Next, the practitioner may withdraw the inner tube 224 through the open end of the outer tube 228, as shown in FIG. 11. Once the inner tube 244 is free of the outer tube 228, the practitioner may loosen and remove the closure 260 from the inner tube 224 and then withdraw the internal implant retainer 264 from within an inner tube 224, as shown in FIG. 12. As shown in FIG. 13, the practitioner may open the internal implant retainer 264 to access the bone fusion implant 100. It is contemplated that the internal implant retainer 264 may be quickly and easily opened by simply unsnapping the push clasps 284 (see FIG. 10) and then unfolding the first half 268 and the second half 272 of the internal implant retainer 264 to gain access the bone fusion plate 100 stored in the retaining cavity 280. Once free of the sterile tube package 220 and the internal implant retainer 264, the sterile bone fusion plate 100 may be implanted in a patient.

Methods for stabilizing and fixating fractures, revision procedures, joint fusion, osteotomies and reconstruction of the small bones of the human body may, in some embodiments, include configuring a bone fusion plate 100 comprising a generally elongate member having an upper surface 104 and a lower, bone contact surface 108. In some embodiments, the methods may include forming a layer structure 128 comprising the bone fusion plate 100. In some embodiments, forming the layer structure 128 may comprise forming a series of layers 132 of carbon fibers and orienting the carbon fibers at different angles. Forming the series of layers 132 of carbon fibers may comprise, in some embodiments, forming layers of PEEK carbon tape or performing a 3D printing process. It is contemplated that orienting the carbon fibers at different angles optimizes mechanical properties, such as bending and fatigue strength, of the bone fusion plate 100.

In some embodiments, the methods may include configuring a plate length 136 that ranges between about 0.90 inches and about 10.00 inches. Further, in some embodiments, configuring the bone fusion plate 100 may comprise configuring a plate width 140 that ranges between about 0.25 inches and about 2.00 inches. Further, in some embodiments, configuring the bone fusion plate 100 may comprise configuring a plate thickness 144 that ranges between about 0.05 inches and about 0.10 inches.

In some embodiments, the methods may include configuring a longitudinal aspect of the bone fusion plate 100 to include one or more angles or bends such that the bone fusion plate 100 matches the anatomy of the bone to be fused. In some embodiments, configuring the bone fusion plate 100 may comprise configuring a vertical angle 164 to accommodate a dorsiflexion angle of the bones in a human foot, the vertical angle 164 ranging between about 2 degrees and about 10 degrees. In some embodiments, configuring the bone fusion plate 100 may comprise configuring a horizontal angle 180 to accommodate a hallux valgus angle of the human foot, the horizontal angle 180 ranging between about 2 degrees and about 10 degrees. Further, in some embodiments, configuring the bone fusion plate 100 may include configuring a transverse aspect of the bone fusion plate 100 to include one or more curvatures such that the bone fusion plate 100 matches the anatomy of the bone to be fused.

The methods may include, in some embodiments, configuring the bone fusion plate 100 may include configuring any of various markings, shapes, diagrams, symbols, words, numbers, or indicators to assist a practitioner with correctly orienting the bone fusion plate 100 onto the bones to be treated. In some embodiments, configuring the bone fusion plate 100 may include disposing a joint line 212 on the upper surface 104 to assist the practitioner with aligning a longitudinal aspect of the bone fusion plate 100 with a joint to be fixated.

The methods may include, in some embodiments, disposing two or more fixation apertures 112 along the bone fusion plate 100 and configuring the two or more fixation apertures 112 to receive bone screws 116. In some embodiments, disposing the two or more fixation apertures 112 comprises disposing 4-5 fixation apertures 112. However, in some embodiments, disposing the two or more fixation apertures 112 may comprise disposing less than 4 or more than 5 fixation apertures 112.

The methods may include, in some embodiments, configuring at least one elongated aperture 124 to receive a bone screw 116. The methods may include, in some embodiments, configuring the elongated aperture 124 to receive the bone screw 116 at an angle of substantially 90° with respect to the plane of the bone fusion plate 100. In some embodiments, configuring the elongated aperture 124 includes configuring the elongated aperture 124 to receive the bone screw 116 at an oblique angle with respect to the plane of the bone fusion plate 100.

The methods may include, in some embodiments, configuring a threaded hole 120 comprising each of the two or more fixation apertures 112. Configuring the threaded hole 120 may, in some embodiments, include disposing the threaded hole 120 below the upper surface 104 of the bone fusion plate 100 to allow a countersunk head of the bone screw 116 to assume a level that is flush with, or disposed below, the upper surface 104 when the fastener is tightened to hold the bone fusion plate 100 against the bone. In some embodiments, configuring the threaded hole 120 includes forming a chamfer angle ranging between about 60 degrees and about 120 degrees with respect to the upper surface 104. Further, in some embodiments, forming the chamfer angle includes forming the chamfer angle at either about 82 degrees or about 90 degrees.

In some embodiments, configuring the threaded hole 120 may include configuring the threaded hole 120 to prevent the bone screw 116 from backing out of the bone after being implanted in a patient. Configuring the threaded hole 120 may include, in some embodiments, configuring the threaded hole 120 to engage with the head of a locking bone screw 116. Configuring the threaded countersink (screw hole) surface 120 may include, in some embodiments, configuring the threaded hole 120 to accept a threaded locking drill guide tower for on-axis pilot hole preparation.

The methods may include, in some embodiments, configuring the bone fusion plate 100 to be shipped and stored in a sterile state. In some embodiments, configuring the bone fusion plate 100 to be shipped and stored in a sterile state includes forming a sterile tube package 220 comprising an inner tube 224 contained inside an outer tube 228. Forming the sterile tube package 220 may include, in some embodiments, configuring the outer tube 228 to receive and retain the inner tube 224, such that a sterile environment within at least the inner tube 224 is maintained during shipping and storage of the fusion bone plate 100. Further, forming the sterile tube package 220 may include, in some embodiments, forming the inner tube 224 and the outer tube 228 of a rigid material that is capable of withstanding sterilization by way of GAMMA Sterilization methods.

In some embodiments, forming the sterile tube package 220 includes configuring the inner tube 224 to house an internal implant retainer 264. The internal implant retainer 264 may be configured, in some embodiments, to protect the bone fusion plate 100 within the inner tube 224 during shipping and storage inside the sterile tube package 220. In some embodiments, configuring the internal implant retainer 264 comprises: forming a first half 268 and a second half 272 that share intervening fold lines 276; and disposing a retaining cavity 280 and one or more push clasps 284 in each of the first half 268 and the second half 272. Forming the first half 268 may include, in some embodiments, configuring the first half 268 to be folded onto to the second half 272, by way of the fold lines 276, to surround the bone fusion plate 100 between the retaining cavities 280. Further, disposing the push clasps 284 may include, in some embodiments, configuring the push clasps 284 to be pressed together, such that the bone fusion plate 100 is enclosed within the retaining cavities 280 for storage and shipping within the sterile package 220.

FIG. 14 illustrates an exemplary embodiment of a bone fusion plate 300 configured for stabilization and fixation of fractures, revision procedures, joint fusion, osteotomies and reconstruction of the bones of the human body, according to the present disclosure. Bone fusion plate 300 comprises a generally elongated member having an upper surface 304 and a lower, bone contact surface 308 (see, for example, FIG. 17). Disposed along the bone fusion plate 300 are two or more fixation apertures 112 (described with respect to FIG. 3) suitable for receiving fasteners, such as bone screws 116 (see FIGS. 6-8). It is contemplated that although 4 fixation apertures 112 are shown in the illustrated embodiment of FIG. 14, in other embodiments, the bone fusion plate 300 may include any number of fixation apertures 112, without limitation.

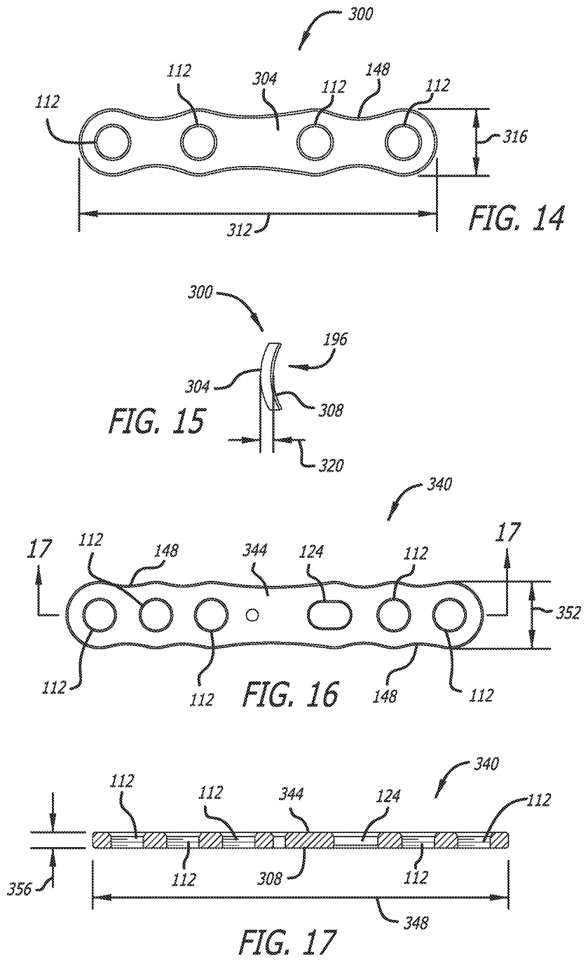

Moreover, the overall dimensions of the bone fusion plate 300 may be varied according to the anatomy of the specific bones to be treated. In some embodiments, for example, the bone fusion plate 300 comprises a plate length 312 that ranges between about 0.90 inches and about 10.00 inches. In some embodiments, the bone fusion plate 300 comprises a plate width 316 that ranges between about 0.25 inches and about 2.00 inches. In some embodiments, the bone fusion plate 100 comprises a plate thickness 320 (see FIG. 15) that ranges between about 0.04 inches and about 0.10 inches. As further shown in FIG. 14, the bone fusion plate 300 can have straight sides or any of various surface features, such as one or more scallops 148 or other similar features that accommodate the anatomy of the specific bone being treated. Further, as best shown in FIG. 15, the bone fusion plate 300 is curved in a direction that is substantially perpendicular to the longitudinal aspect of the plate, giving rise to a transverse curvature 196. As will be appreciated, the transverse curvature 196 may be adapted to accommodate the specific anatomy of the bones to be fused.

FIGS. 16-17 illustrate an exemplary embodiment of a bone fusion plate 340 that is configured for stabilization and fixation of fractures, revision procedures, joint fusion, osteotomies and reconstruction of the bones of the human body, according to the present disclosure. Bone fusion plate 340 comprises a generally elongated member having an upper surface 344 and a lower, bone contact surface 308. Disposed along the bone fusion plate 340 are two or more fixation apertures 112 (described with respect to FIG. 3) configured for receiving fasteners, such as bone screws 116 (see FIGS. 6-8). As described herein, although 5 fixation apertures 112 are shown in the illustrated embodiment of FIG. 16, in other embodiments, the bone fusion plate 340 may include any number of fixation apertures 112, without limitation.

As shown in FIGS. 16-17, the bone fusion plate 340 preferably comprises at least one elongated aperture 124 configured to receive a threaded fastener, such as a bone screw 116 (see FIGS. 6-8). As described hereinabove, the elongated aperture 124 may receive the bone screw 116 at an angle of substantially 90° with respect to the plane of the bone fusion plate 340, similar to the fixation apertures 112. Unlike the fixation apertures 112, however, the elongated aperture 124 may further receive the bone screw 116 at an oblique angle with respect to the plane of the bone fusion plate 340. As will be appreciated, the oblique angle of the bone screw 116 facilitates compressing adjacent bone portions together so as to encourage bone fusion.

With continuing reference to FIGS. 16-17, the overall dimensions of the bone fusion plate 340 may be varied according to the anatomy of the specific bones to be treated. For example, in some embodiments the bone fusion plate 340 comprises a plate length 348 that ranges between about 0.90 inches and about 10.00 inches. Further, the bone fusion plate 340 may, in some embodiments, comprise a plate width 352 that ranges between about 0.25 inches and about 2.00 inches. In some embodiments, the bone fusion plate 340 may comprise a plate thickness 356 that ranges between about 0.04 inches and about 0.10 inches. As further shown in FIG. 16, the bone fusion plate 340 can have straight sides or any of various surface features, such as one or more scallops 148 or other similar features that accommodate the anatomy of the specific bone being treated. Further, the bone fusion plate 340 can include a transverse curvature, such as the transverse curvature 196 discussed with respect to FIG. 15, or the bone fusion plate 340 may be generally planar as shown in FIG. 17 to accommodate the specific anatomy of the bones to be fused.

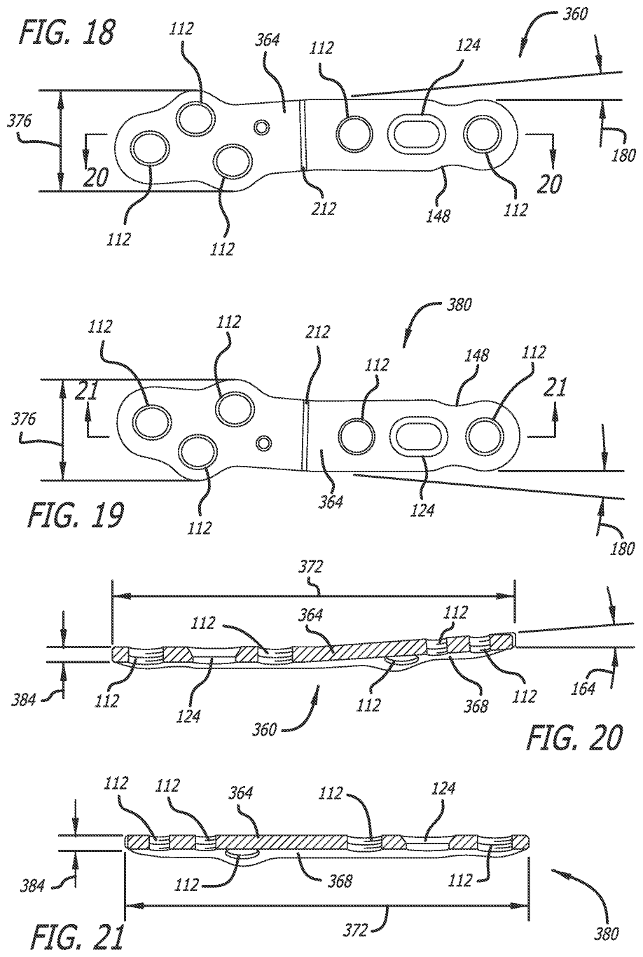

FIGS. 18-21 illustrate bone fusion plates that are configured to accommodate a proper hallux valgus angle of a patient's feet. More specifically, FIG. 18 illustrates an exemplary embodiment of a bone fusion plate 360 configured to accommodate a proper hallux valgus angle of a patient's left foot, while FIG. 19 illustrates an exemplary embodiment of a bone fusion plate 380 configured to accommodate a proper hallux valgus angle of a patient's right foot, in accordance with the present disclosure. As such, it should be understood that the bone fusion plate 360 is substantially similar to the bone fusion plate 380, with the exception that the bone fusion plate 360 is configured specifically to operate with a left foot and the bone fusion plate 380 is configured specifically to operate with a right foot. In particular, the bone fusion plates 360, 380 are horizontally curved in opposite directions, giving rise to a horizontal angle 180 relative to a longitudinal aspect of each plate. As will be recognized, the horizontal angle 180 is adapted to accommodate the hallux valgus angle of the human foot, and thus the horizontal angle 180 may range between about 2 degrees and about 10 degrees, without limitation.

Bone fusion plates 360, 380 comprise generally elongated members each having an upper surface 364 and a lower, bone contact surface 368 (see FIGS. 20-21). Disposed along each of the bone fusion plates 360, 380 are two or more fixation apertures 112 (described with respect to FIG. 3) configured for receiving fasteners, such as bone screws 116 (see FIGS. 6-8). It should be understood that although 5 fixation apertures 112 are shown in the embodiments of FIGS. 18-19, in other embodiments, the bone fusion plate 360, 380 may include any number of fixation apertures 112, without limitation. Further, each of the bone fusion plates 360, 380 preferably includes at least one elongated aperture 124 configured to receive a threaded fastener, such as a bone screw 116. As described hereinabove, the elongated aperture 124 may receive the bone screw 116 at an angle of substantially 90° with respect to the plane of the bone fusion plate 360 (380), similar to the fixation apertures 112. Unlike the fixation apertures 112, however, the elongated aperture 124 may further receive the bone screw 116 at an oblique angle with respect to the plane of the bone fusion plate 360 (380). As will be appreciated, the oblique angle of the bone screw 116 facilitates compressing adjacent bone portions together so as to encourage bone fusion.

Similar to other bone fusion plates disclosed herein, the overall dimensions of the bone fusion plates 360, 380 may be varied according to the anatomy of the specific bones to be treated. For example, in some embodiments the bone fusion plates 360, 380 each comprises a plate length 372 (see FIGS. 20-21) ranging between about 0.90 inches and about 10.00 inches. Further, the bone fusion plates 360, 380 may, in some embodiments, each comprise a plate width 376 ranging between about 0.25 inches and about 2.00 inches. In some embodiments, the bone fusion plates 360, 380 may each comprise a plate thickness 384 (see FIGS. 20-21) ranging between about 0.04 inches and about 0.10 inches. As further shown in FIGS. 18-19, the bone fusion plates 360, 380 can have straight sides or any of various surface features, such as one or more scallops 148 or other similar features that accommodate the anatomy of the specific bone being treated.

As shown in FIGS. 18-19, a longitudinal aspect of the bone fusion plates 360, 380 can be characterized by one or more angles or bends such that the bone fusion plate 360, 380 match the anatomy of the bones to be fused. For example, as shown in FIG. 20, bone fusion plate 360 may include a vertical angle 164 that is adapted to fixate a joint 152 between a first bone 156 and a second bone 160, as shown in FIG. 5.

As will be appreciated, vertical angle 164 may range between about 2 degrees and about 10 degrees so as to accommodate a dorsiflexion angle of the bones in a human foot. In another example, shown in FIG. 21, the bone fusion plate 380 is generally straight along the longitudinal aspect of the bone fusion plate 380. As such, the bone fusion plate 380 may be used in cases wherein a dorsiflexion angle of the bones is contraindicated.

Moreover, the bone fusion plates 360, 380 are not limited to bends or angles along the longitudinal aspect of the plate, but rather the bone fusion plates 360, 380 may include curvature along a transverse aspect of the bone fusion plates 360, 380. For example, in some embodiments, the bone fusion plates 360, 380 may be fastened across a joint 184 between a first bone 188 and a second bone 192 by way of multiple bone screws 116, as shown in FIG. 7. In such embodiments, the bone fusion plates 360, 380 may include a transverse curvature 196 (see FIG. 15) that is configured to accommodate the specific anatomy of the bones to be fused.

It should be borne in mind that the bone fusion plates 360, 380 are not limited to the specific angles and/or curvatures shown in the figures, but rather any combination of curvatures and/or angles may be disposed along the longitudinal and transverse aspects of the bone fusion plates 360, 380, without limitation. In some embodiments, the curvatures incorporated into the longitudinal and transverse aspects of the bone fusion plates 360, 380 may be configured to change as a function of distance from a midline of each of the bone fusion plates 360, 380.

In some embodiments, the curvatures and/or angles along the longitudinal and transverse aspects of the bone fusion plates 360, 380 may be configured to match a specific anatomy of the bones to be treated. As will be appreciated, a bone fusion plate that is “anatomy specific” must be correctly fastened to the bones to be treated. To this end, the bone fusion plates 360, 380 may include any of various markings, shapes, diagrams, symbols, words, numbers, or indicators configured to assist a practitioner with correctly orienting the bone fusion plates 360, 380 onto the bones to be treated. For example, in the illustrated embodiments of FIGS. 18-19, the bone fusion plates 360, 380 each includes a joint line 212 on the upper surface 364 of the plate. As described herein with respect to FIG. 8, the joint line 212 assists the practitioner with aligning the longitudinal aspect of the bone fusion plate 360 (380) with a joint, thereby ensuring that any curvatures and/or angles comprising the bone fusion plate 360 (380) substantially match the specific anatomy of the bones to be fused. Thus, it is contemplated that the joint line 212 simplifies accurately matching the curvatures and/or angles comprising the bone fusion plates 360, 380 with the anatomy of the bones being treated.

While the PEEK carbon bone fusion plate and methods have been described in terms of particular variations and illustrative figures, those of ordinary skill in the art will recognize that the PEEK carbon bone fusion plate is not limited to the variations or figures described. In addition, where methods and steps described above indicate certain events occurring in certain order, those of ordinary skill in the art will recognize that the ordering of certain steps may be modified and that such modifications are in accordance with the variations of the PEEK carbon bone fusion plate. Additionally, certain of the steps may be performed concurrently in a parallel process, when possible, as well as performed sequentially as described above. To the extent there are variations of the PEEK carbon bone fusion plate, which are within the spirit of the disclosure or equivalent to the PEEK carbon bone fusion plate found in the claims, it is the intent that this patent will cover those variations as well. Therefore, the present disclosure is to be understood as not limited by the specific embodiments described herein, but only by scope of the appended claims.

What is claimed is:

1. An apparatus for stabilizing and fixating fractures, revision procedures, joint fusion, osteotomies and reconstruction of the bones of the human body, comprising:
a bone fusion plate comprising a generally elongate member having an upper surface and a lower, bone contact surface;
a layer structure comprising a series of layers of PEEK-embedded carbon fiber tape oriented at different angles;
two or more fixation apertures disposed along the bone fusion plate and configured to receive bone screws; and
a threaded hole comprising each of the two or more fixation apertures;
wherein a plate length ranges between about 0.90 inches and about 10.00 inches, a plate width ranges between about 0.25 inches and about 2.00 inches, and a plate thickness ranges between about 0.04 inches and about 0.10 inches.

2. The apparatus of claim 1, wherein the threaded hole is configured to accept a threaded locking drill guide tower for on-axis pilot hole preparation.

3. The apparatus of claim 1, wherein the threaded hole is configured to accept a threaded locking bone screw or a non-threaded bone screw.

4. The apparatus of claim 1, wherein the PEEK-embedded carbon fiber tape includes an additional additive that provides visualization of both the fusion site and the bone plate under X-Ray.

5. The apparatus of claim 1, wherein a longitudinal aspect of the bone fusion plate includes one or more angles or bends such that the bone fusion plate matches the anatomy of the bone to be fused.

6. The apparatus of claim 1, wherein the bone fusion plate includes a vertical angle configured to accommodate a dorsiflexion angle of the bones in a human foot.

7. The apparatus of claim 1, wherein the bone fusion plate includes a horizontal angle configured to accommodate a hallux valgus angle of the human foot ranging between about 2 degrees and about 10 degrees.

8. The apparatus of claim 1, wherein a transverse aspect of the bone fusion plate includes one or more curvatures such that the bone fusion plate matches the anatomy of the bone to be fused.

9. The apparatus of claim 1, wherein the bone fusion plate includes any of various markings, shapes, diagrams, symbols, words, numbers, or indicators configured to assist a practitioner with correctly orienting the bone fusion plate onto the bones to be treated.

10. The apparatus of claim 9, wherein the bone fusion plate includes a joint line indicator disposed on the upper surface to assist the practitioner with aligning a longitudinal aspect of the bone fusion plate with a joint to be fixated.

11. The apparatus of claim 1, wherein the bone fusion plate is configured to be shipped and stored in sterile packaging in a single barrier or dual barrier configuration.

12. The apparatus of claim 11, wherein the sterile packaging is a tube assembly comprising an inner tube contained inside an outer tube.

13. The apparatus of claim 12, wherein the inner tube is configured to house an internal implant retainer that is configured to protect the bone fusion plate within the sterile packaging during shipping and storage.

14. The apparatus of claim 13, wherein the internal implant retainer comprises: a first half and a second half that share intervening fold lines; and a retaining cavity and one or more push clasps disposed in each of the first half and the second half.

15. The apparatus of claim 14, wherein the first half is configured to be folded onto to the second half, by way of the fold lines, to surround the bone fusion plate between the retaining cavities; and wherein the push clasps are configured to be pressed together, such that the bone fusion plate is enclosed within the retaining cavities for storage and shipping within the sterile package.

16. The apparatus of claim 11, wherein the sterile packaging is a thermoform tray assembly comprising an inner tray contained inside an outer tray.

17. The apparatus of claim 11, wherein the sterile packaging is a pouch assembly comprising an inner pouch contained inside an outer pouch.

18. The apparatus of claim 11, wherein the sterile packaging is capable of withstanding sterilization by way of GAMMA sterilization.

19. The apparatus of claim 11, wherein the sterile packaging is capable of withstanding sterilization by way of Ethylene Oxide Sterilization.

20. The apparatus of claim 11, wherein the sterile packaging is capable of withstanding sterilization by way of Steam sterilization.

21. The apparatus of claim 11, wherein the sterile packaging is capable of withstanding sterilization by way of Electron Beam sterilization.
